(19) United States
(12) Patent Application Publication
(10) Pub. No.: US 2022/0193442 A1
(43) Pub. Date: Jun. 23, 2022

(54) SYSTEMS FOR FACILITATING LOW-LEVEL LASER THERAPY ON A HEAD OF A USER

(71) Applicant: Nolan A. White, Logan, UT (US)

(72) Inventor: Nolan A. White, Logan, UT (US)

(21) Appl. No.: 17/558,127

(22) Filed: Dec. 21, 2021

Related U.S. Application Data
(60) Provisional application No. 63/129,214, filed on Dec. 22, 2020.

(51) Int. Cl. A61N 5/067; A61N 5/06

(52) U.S. Cl. CPC A61N 5/067; A61N 5/0613; A61N 2005/0647; A61N 2005/0658; A61N 2005/0632; A61N 2005/0651

(57) ABSTRACT

A system configured for facilitating LLLT on a head of a user includes an LLLT system and a head-mounting device. The LLLT system includes a laser diode lead configured to output laser light suitable for LLLT and a laser controller configured to provide power to and control operation of the laser diode lead. The head-mounting device is wearable on a head of a user, and the head-mounting device comprising an attachment point. The attachment point is configured to secure the laser diode lead to direct laser light output by the laser diode lead toward a treatment area on a head of a user.

### CROSS-REFERENCE TO RELATED APPLICATIONS

This application claims priority to and the benefit of U.S. Provisional Patent Application No. 63/129,214, filed Dec. 22, 2020 and titled “Systems for Facilitating Low-Level Laser Therapy on a Head of a User”, the entirety of which is incorporated herein by this reference.

### BACKGROUND

A growing body of research has shown the effectiveness of low-level laser therapy (LLLT) for providing various therapeutic effects and treating a variety of medical conditions. Various demonstrated mechanisms of LLLT include, but are not limited to, increasing ATP production and cellular energy production by activating cytochrome C oxidase enzyme, suppressing inflammation and inflammatory cytokines, increasing cellular antioxidant pathway production, reducing effects of free radical damage, decreasing oxidative stress in the brain, increasing stem cell production, increasing growth factors for cells (e.g., NGF, BDNF, IGF-1, and VEGF), preventing neuronal death by facilitating greater membrane stability and resistance to depolarization, increasing natural bodily opioids, increasing mitochondria biogenesis, promoting the synthesis of DNA and RNA, increasing blood flow and circulation, down-regulating glial priming, decreasing amyloid beta burden, restoring axonal transport from tau hyperphosphorylation, speeding up metabolism, stimulating repair and/or regeneration of damaged cells, causing fat cells to release stored fat into the blood stream, decreasing stress hormones, increasing HGH, activating genes that regenerate and repair cell and DNA damage, and/or others.

Accordingly, medical practitioners implement LLLT in a variety of ways to treat a variety of patient conditions and/or to improve patient health. By way of non-limiting example, LLLT is used by medical practitioners to treat and/or stimulate patient healing from back, neck and/or other musculoskeletal pain. Medical practitioners also perform LLLT on patient brain tissue to facilitate neurorehabilitation (e.g., to facilitate recovery from stroke, degenerative or traumatic brain disorders/injuries) and/or to optimize brain function and general health. Many patients have come to realize the benefits of LLLT, thereby increasing patient demand for regular LLLT sessions.

However, many conventional LLLT devices for use by medical practitioners rely on manual operation/manipulation of a laser diode lead by a medical practitioner to direct LLLT to desired treatment areas for desired treatment time periods. Such configurations often restrict the ability of medical practitioners to treat/handle multiple patients. Furthermore, such configurations may reduce treatment efficiency by increasing the amount of time that medical practitioners must spend manually manipulating a laser diode lead to facilitate LLLT. Additionally, such configurations may limit the ability of lay users to operate LLLT devices for self-care.

Still furthermore, although some LLLT devices include a stand for directing laser light toward a portion of a user's body in a static manner, such LLLT devices limit the ability of users to locomote or perform other bodily actions while receiving LLLT. Such LLLT devices therefore fail to facilitate LLLT on bodily cells in dynamic or active states, such as during user locomotion or action.

Accordingly, there exists a need for improved systems for facilitating LLLT on various parts of patient bodies.

The subject matter claimed herein is not limited to embodiments that solve any disadvantages or that operate only in environments such as those described above. Rather, this background is only provided to illustrate one exemplary technology area where some embodiments described herein may be practiced.

### BRIEF SUMMARY

Embodiments of the present disclosure are directed to systems for facilitating LLLT on a head of a user.

In one aspect, a system configured for facilitating LLLT on a head of a user includes an LLLT system and a head-mounting device. The LLLT system includes a laser diode lead configured to output laser light suitable for LLLT and a laser controller configured to provide power to and control operation of the laser diode lead. The head-mounting device is wearable on a head of a user, and the head-mounting device includes an attachment point. The attachment point is configured to secure the laser diode lead to the head-mounting device and to direct laser light output by the laser diode lead toward a treatment area on a head of a user. A location and a shape of the attachment point(s) influences the direction of laser light output by the laser diode lead.

In some implementations, laser light output by the LLLT system is configured for penetrating through a skull of the user to reach brain tissue of the user. In some instances, the LLLT system is configured to output laser light including one or more wavelengths within the near-infrared region (e.g., including one or more wavelengths within a range of about 780 nm to about 840 nm and/or including one or more wavelengths within a range of about 840 nm to 2500 nm). In some instances, the treatment area on the head of the user includes a targeted treatment area to focus LLLT on certain brain tissue, such as a treatment area of a size that is less than 50% of an area of a scalp of the user.

In some implementations, the head-mounting device includes any number of attachment points, and the attachment point(s) are configured to selectively and removably secure the laser diode lead to the head-mounting device. In some instances, the LLLT system includes a plurality of laser diode leads, and multiple attachment points of the head-mounting device are configured to simultaneously secure separate laser diode leads to facilitate simultaneous LLLT on separate respective treatment areas on the head of the user. In some instances, securing the separate laser diode leads in multiple attachment points enables a user to direct the laser light emitted by the laser diode leads to a desired treatment area.

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

Additional features and advantages of the disclosure will be set forth in the description which follows, and in part will be obvious from the description, or may be learned by the practice of the disclosure. The features and advantages of the disclosure may be realized and obtained by means of the instruments and combinations particularly pointed out in the appended claims. These and other features of the present disclosure will become more fully apparent from the following description and appended claims or may be learned by the practice of the disclosure as set forth hereinafter.

### BRIEF DESCRIPTION OF THE DRAWINGS

In order to describe the manner in which the above recited and other advantages and features of the disclosure can be obtained, a more particular description of the disclosure briefly described above will be rendered by reference to specific embodiments thereof, which are illustrated in the appended drawings. It is appreciated that these drawings depict only typical embodiments of the disclosure and are not therefore to be considered to be limiting of its scope.

The disclosure will be described and explained with additional specificity and detail through the use of the accompanying drawings in which:

FIGS. 1 and 2 illustrate examples of LLLT systems;

FIG. 3 illustrates example laser light emitted by an LLLT system;

FIGS. 4A-4D illustrate examples of a head-mounting device;

FIGS. 5A-5D illustrate example systems configured for facilitating LLLT on a head of a user;

FIGS. 6 and 7 illustrate example implementations of a system configured for facilitating LLLT on a head of a user where the system includes an additional mounting device; and

FIG. 8 illustrates a conceptual representation of a system configured for facilitating LLLT on a head of a user where the entire LLLT system becomes mounted on the head-mounting device.

### DETAILED DESCRIPTION

Before describing various embodiments of the present disclosure in detail, it is to be understood that this disclosure is not limited to the parameters of the particularly exemplified systems, methods, apparatus, products, processes, and/or kits, which may, of course, vary. Thus, while certain embodiments of the present disclosure will be described in detail, with reference to specific configurations, parameters, components, elements, etc., the descriptions are illustrative and are not to be construed as limiting the scope of the claimed invention. In addition, any headings used herein are for organizational purposes only, and the terminology used herein is for the purpose of describing the embodiments. Neither are not meant to be used to limit the scope of the description or the claims.

Embodiments of the present disclosure are directed to systems for facilitating LLLT on a head of a user. In some embodiments, a system configured for facilitating LLLT on a head of a user includes an LLLT system and a head-mounting device. The LLLT system includes a laser diode lead configured to output laser light suitable for LLLT and a laser controller configured to provide power to and control operation of the laser diode lead. The head-mounting device is wearable on a head of a user, and the head-mounting device includes an attachment point. The attachment point is configured to secure the laser diode lead to the head-mounting device and to direct laser light output by the laser diode lead toward a treatment area on a head of a user.

Those skilled in the art will appreciate, in view of the present disclosure, that at least some of the disclosed embodiments may facilitate LLLT on a head of users in an advantageous manner. For example, systems of the present disclosure provide a head-mounting device with one or more laser diode leads implemented therein, thereby allowing users to wear the head-mounting device on their head to facilitate LLLT on their head in a passive manner. The laser diode lead(s) may be configured to output laser light that penetrates through a user skull to allow for LLLT of user brain tissue. Such laser light may be configured for various therapeutic and/or health-promoting uses, such as to facilitate recovery from stroke or degenerative or traumatic brain disorders/injuries and/or to optimize brain function.

Facilitating passive LLLT via a head-mounting device as described herein may enable medical practitioners to provide LLLT services in an advantageous and efficient manner. For instance, rather than needing to manually manipulate a laser diode lead to administer LLLT on a patient's head, a medical practitioner may determine appropriate treatment settings for a particular patient (e.g., treatment area on the patient's head, laser power, treatment duration, etc.) and place a head-mounting device with LLLT laser diode leads on the head of the patient. Thus, the patient may then undergo passive LLLT on their head without requiring the medical practitioner to manually administer the LLLT, allowing the medical practitioner to tend to other duties during the passive LLLT session (e.g., perhaps allowing the medical practitioner to initiate a separate passive LLLT session for a separate patient, thereby improving treatment efficiency and capacity for the medical practitioner).

Furthermore, in some instances, providing systems for passive LLLT may enable users to self-administer LLLT in a safe manner. For instance, a lay user may obtain a system of the present disclosure for facilitating passive LLLT on the head and may operate the LLLT system using prescribed settings to allow for safe and readily available LLLT to meet the demand of the patient. In some instances, the prescribed settings are provided by a medical practitioner (e.g., under remote medical care circumstances, such as where in-person meetings with medical practitioners are limited). Such prescribed settings may include laser diode lead placement on the head-mounting device, laser power, treatment duration, and/or others.

Still furthermore, systems of the present disclosure may allow users to move around or perform other bodily actions while receiving LLLT. Beneficially, systems of the present disclosure may facilitate LLLT on brain tissue while the brain tissue is in multiple states (e.g., while actively transmitting signals to cause bodily movements). Such functionality may broaden the applicability and/or effectiveness of LLLT for facilitating neurorehabilitation and/or improving brain health/function. By way of non-limiting example, systems of the present disclosure may be implemented to improve various aspects of user health and/or performance, such as improving performance in athletic activities (e.g., baseball, tennis, basketball, golf, running events, snowboarding, skiing, cycling, mountain biking, football, weightlifting, martial arts, soccer), improving performance in cognitive activities (e.g., video gaming, strategic activities/games, workplace activities/training, music performance, flight training), pre-participation concussion prevention for athletic activities, addressing military pre-trauma, facilitating rehabilitation (e.g., post-surgery), improving disability or mental decline symptoms, providing relief in occupations where brain fatigue is common, etcetera.

In addition, at least some head-mounting devices of the present disclosure may be configured to receive laser diode leads of conventional LLLT systems that are not specially configured or manufactured for use with wearable systems. For instance, many medical practitioners (e.g., chiropractors) have existing LLLT systems that include hand-controllable laser diode leads that allow medical practitioners to administer LLLT manually. At least some head-mounting devices of the present disclosure may be configured to removably secure such conventional LLLT laser diode leads (e.g., laser diode leads that are originally manufactured or configured for the administration of LLLT in a hand-controlled fashion) to allow existing LLLT systems to be advantageously used for passive LLLT on heads of patients.

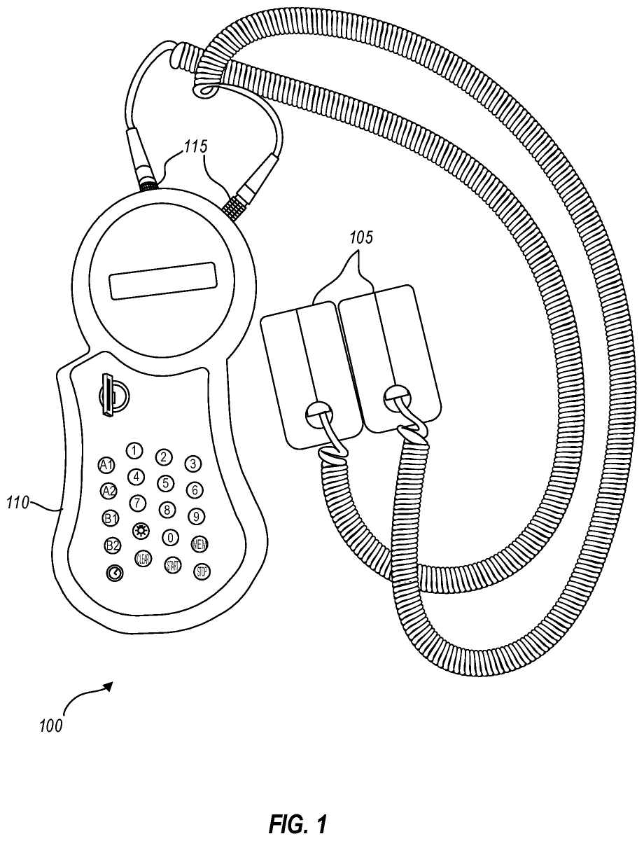

Having described some of the various high-level features and benefits of the disclosed embodiments, attention will now be directed to FIGS. 1-8. These Figures illustrate various conceptual representations, architectures, methods, and/or supporting illustrations related to the disclosed embodiments.

FIG. 1 illustrates example components of a system configured for facilitating LLLT on a head of a user, in accordance with the present disclosure. In particular, FIG. 1 illustrates an example of an LLLT system 100 that may be part of a system for facilitating LLLT on a head of a user. As is illustrated in FIG. 1, the LLLT system 100 includes a plurality of laser diode leads 105, which are configured to output laser light suitable for LLLT.

FIG. 1 also illustrates that the LLLT system 100 includes a laser controller 110. FIG. 1 shows the laser controller 110 as connected to the laser diode leads 105 (e.g., via plugs 115) to enable the laser controller 110 to provide power to and/or otherwise control operation of the laser diode leads 105. By way of non-limiting example, the laser controller 110 may comprise a power supply (e.g., driven by a battery or other voltage source) for powering the laser diodes of the laser diode leads 105 and may comprise electronic circuitry for activating the laser diodes in a pulsed and/or continuous manner (e.g., for predetermined time periods) and/or controlling the laser power of the laser diodes to facilitate LLLT.

Although FIG. 1 illustrates an LLLT system 100 that includes two laser diode leads 105, one will appreciate, in view of the present disclosure, that an LLLT system 100 may include any number of laser diode leads 105 (e.g., one or more than two laser diode leads 105, for example three, five, or seven laser diode leads 105). Furthermore, in some instances, as shown in FIG. 1, the number and/or type of laser diode leads 105 of an LLLT system may be selectively modifiable by a user. For instance, FIG. 1 shows that the laser diode leads 105 are selectively connectable to the laser controller 110 via plugs 115, thereby allowing users to modify the number and/or type of laser diode leads 105 to meet various LLLT purposes (e.g., different laser diode leads 105 may include different optics and/or laser types to facilitate different types of LLLT).

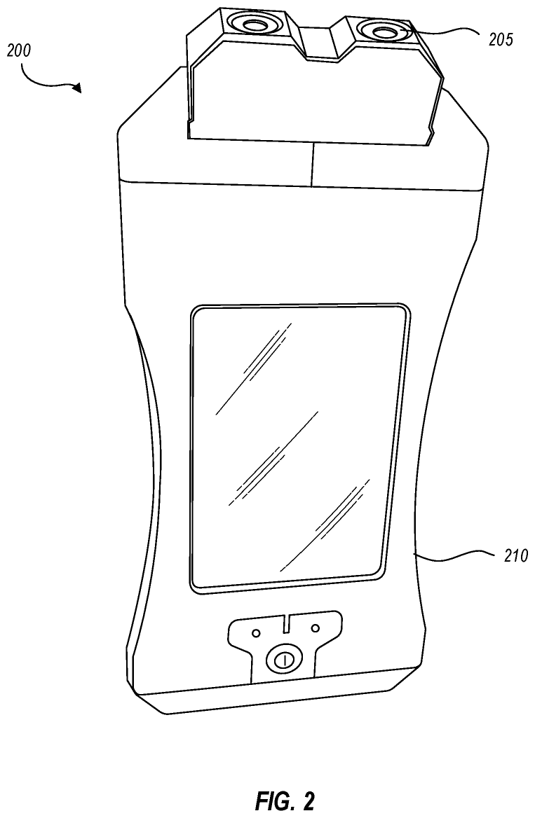

In other instances, the laser diode leads and the laser controller of an LLLT system are not formed as separate or separable units as shown in FIG. 1. For example, FIG. 2 illustrates another example of an LLLT system 200 that may be implemented in a system configured for facilitating LLLT on a head of a user, as described herein. The LLLT system 200 includes laser diode leads 205 and a laser controller 210 that are manufactured or configured as a single unit such that medical practitioners are able to manipulate the positioning of the laser diode leads 205 and the laser controller 210 as a single unit relative to a patient's body to facilitate LLLT.

Those skilled in the art will recognize, in view of the present disclosure, that the particular type or form of the LLLT systems 100 and 200 of FIGS. 1 and 2, respectively, is provided by way of example only and is not limiting of the present disclosure. For instance, the LLLT systems 100 and 200 of FIGS. 1 and 2, respectively, include hand-manipulable laser diode leads that are configured or manufactured for use by medical practitioners to manually direct laser light for LLLT to desired treatment areas for patients (and for desired treatment time periods). Thus, in some instances, an LLLT system of the present disclosure for facilitating LLLT on a head of a user may include a conventional LLLT system that is not specially manufactured for mounting to a head-mounted device (as will be described in more detail with reference to subsequent Figures). In this way, conventional LLLT systems (e.g., which may already be possessed by medical practitioners) may advantageously be implemented for practicing one or more embodiments of the present disclosure, thereby reducing the amount of specialized equipment needed to practice the disclosed embodiments.

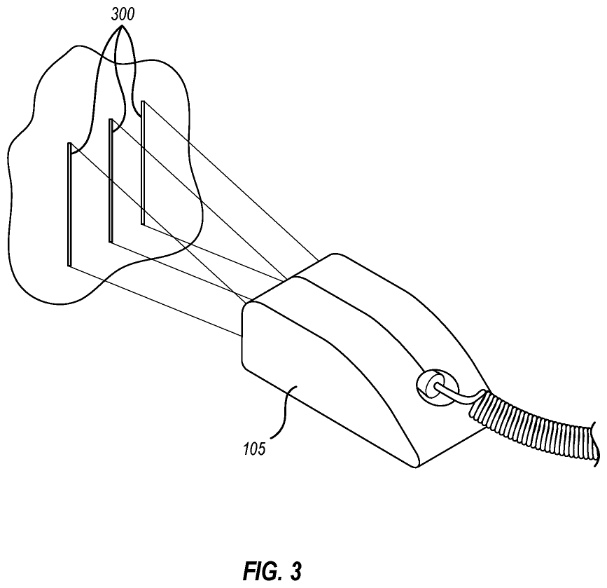

FIG. 3 illustrates example laser light 300 emitted by a laser diode lead 105 of an LLLT system 100. As is evident from FIG. 3, in some instances, the laser light 300 emitted by a laser diode lead for implementation on a system configured for facilitating LLLT on a head of a user includes collimated and/or coherent laser light that is able to stimulate cellular activity in accordance with LLLT. In some implementations, the laser light 300 is able to penetrate through a skull of a user to thereby reach brain tissue of the user. For example, to facilitate penetration through the skull, the laser light 300 may include one or more wavelengths within the near-infrared region (e.g., within a range of about 780 nm to about 2500 nm), such as wavelengths within a range of about 780 nm to about 840 nm. In some embodiments, the laser light 300 may include wavelengths within a range of about 640 nm to about 905 nm. One will appreciate, in view of the present disclosure, that the laser light 300 may include additional or alternative wavelengths, such as wavelengths within a range of about 400 nm to about 700 nm (e.g., 405 nm, 635 nm, and/or others).

In this regard, the laser light 300 may be configured for use to facilitate neurorehabilitation (e.g., to recover from a stroke or degenerative or traumatic brain disorders) or to otherwise optimize brain function and communication throughout the body. Furthermore, in this regard, the laser light 300 described herein may be distinct from laser light used to facilitate functions that do not involve penetrating the skull to reach brain tissue (e.g., hair regrowth). For example, a laser diode lead of the present disclosure may omit light sources that are not configured to penetrate through the skull to facilitate LLLT of brain tissue (e.g., LED light sources and/or laser diodes that are not configured to emit near-infrared light).

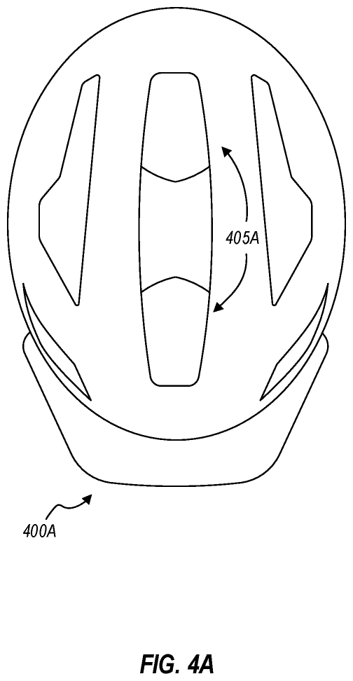

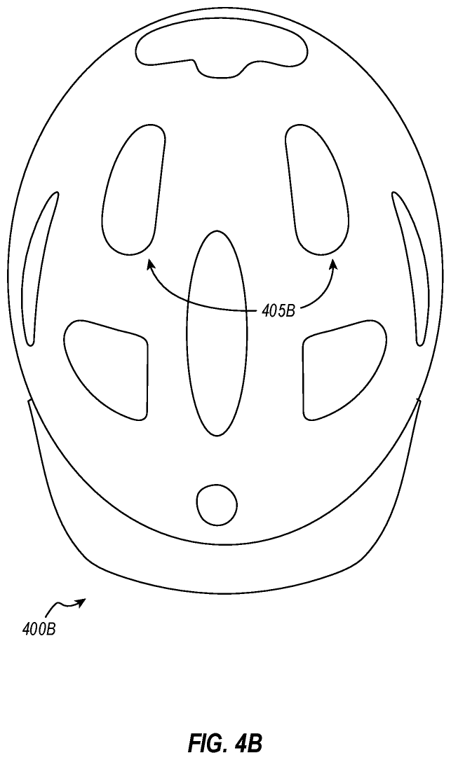

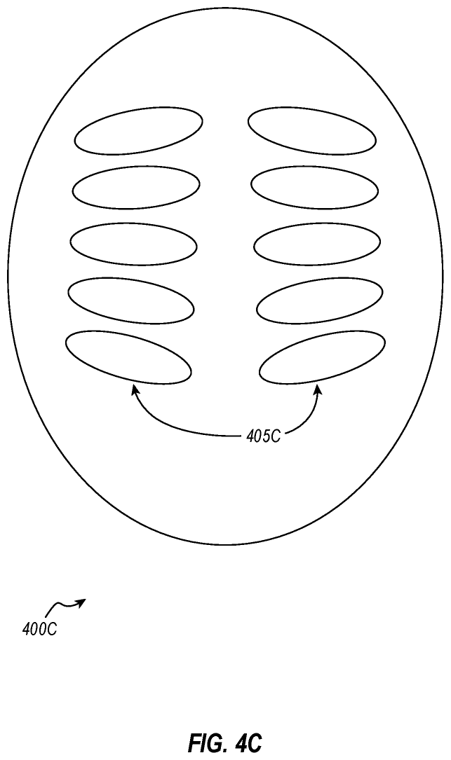

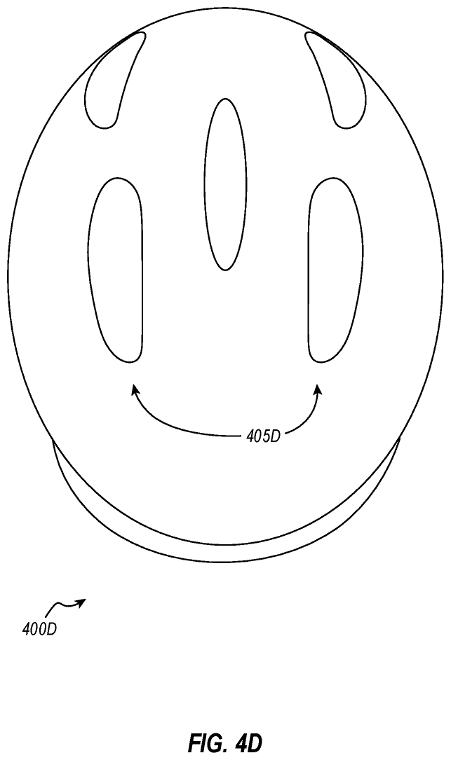

FIGS. 4A-4D illustrate examples of various head-mounting devices 400A-400D of a system configured for facilitating LLLT on a head of a user. The description below with respect to FIG. 4A similarly applies to FIGS. 4B-4D which illustrate additional or alternative embodiments of head-mounting devices 400B-400D and attachment points 405B-405D. FIG. 4A shows that the head-mounting device 400A includes attachment points 405A that are configured to secure a laser diode lead (e.g., laser diode lead 105 or 205). Although FIG. 4A illustrates five attachment points 405A, one will appreciate, in view of the present disclosure, that a head-mounting device 400 may include any number of attachment points 405A (e.g., one, two or three attachment points 405A or more than five attachment points 405A). FIGS. 4B-4D illustrate other shapes, configurations and embodiments of the head-mounting devices 400B-400D and attachment points 405B-405D.

The attachment points 405 are able to secure laser diode leads in a manner that allows laser light emitted by the laser diode lead to be directed toward a treatment area on a head of a user when the user wears the head-mounting device 400 on their head. For example, the head-mounting device 400 is illustrated in FIGS. 4A-4D as implementing the attachment points 405 in the form of slots that are sized to receive a laser diode lead. The slots may receive one or more laser diode leads (e.g., laser diode leads 105) and retain the laser diode leads therein via the walls that form the slots and gravitational forces. For example, the attachment points 405 may retain the laser diode lead(s) by friction or compression fit. In some implementations, a laser diode lead may be readily removable from the slots (or other form of attachment points 405) to allow users to rapidly and/or efficiently reconfigure laser diode leads relative to the head-mounting device 400 in a versatile manner (e.g., to facilitate different LLLT techniques that may include different numbers or positionings of laser diode leads to reach different numbers or locations of treatment areas on a user's head). In this regard, the attachment points 405 may be configured to selectively and removably secure one or more laser diode leads to a head-mounting device 400.

As shown in FIGS. 4A-4D, attachment points 405 may be located on multiple surfaces and sides of the head-mounting device 400. FIG. 4A illustrates an embodiment where rectangular slots or attachment points 405A are disposed on a top or crown surface of the head-mounting device 400A. Attachment points 405 may also be disposed on a front or fore-head surface as well as on a back or occipital surface of the head-mounting device 400. The attachment points 405 disposed on the back or occipital surface of the head-mounting device 400 may be disposed on an upper or lower portion of the back or occipital surface of the head-mounting device 400. Similarly, attachment points 405 may be disposed on side surfaces or a perimeter of the head-mounting device 400, such as right above where a user's ears might fit in the head-mounting device. The attachment points 405 disposed on the perimeter of the head-mounting device 400 may be disposed on an upper or lower portion of the perimeter of the head-mounting device 400. As shown in FIGS. 4A-4D, attachment points 405 are rectangular, ovular or a mixture of multiple shapes; however, attachment points 405 may be any shape suitable for holding laser diode leads in place and directing laser light output by the laser diode leads to a desired treatment area. In some embodiments, attachment points 405 may be customized to particular laser diode leads.

In addition, or as an alternative, to slots, attachment points 405 of a head-mounting device 400 may include any type or number of mechanical devices or configurations operable to facilitate removable securement between a laser diode lead and the head-mounting device 400, such as, by way of non-limiting example, straps (or other tensioning elements), latches, magnet elements, hooks, clips, hook and loop fasteners, threaded elements, and/or any type of interlocking or interconnecting parts.

For illustrative purposes, FIG. 4A shows the head-mounting device 400 implemented as a conventional helmet (e.g., a bicycle helmet or other general-purpose helmet). However, one will appreciate, in view of the present disclosure, that a head-mounting device 400 may take on various forms in accordance with the present disclosure. For example, a head-mounting device 400 may take on the form of a sport helmet (e.g., football or baseball helmet), baseball cap, beanie, or other type of hat, or may be implemented as any type of head-wearable device that is able to receive and secure a laser diode lead of an LLLT system in accordance with the present disclosure (e.g., where the head-wearable device is specifically manufactured to include such securing functionality or where such securing functionality is incidentally present on the head-wearable device despite the head-wearable device not being specifically manufactured to provide such functionality).

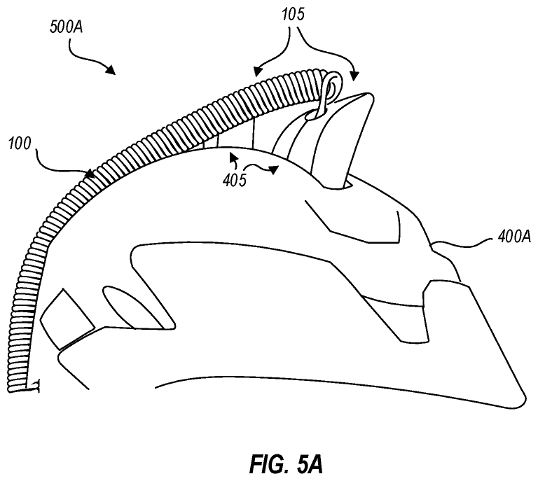

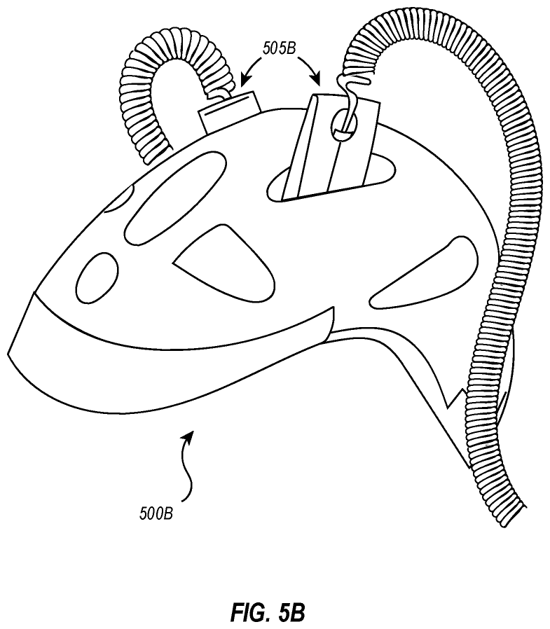

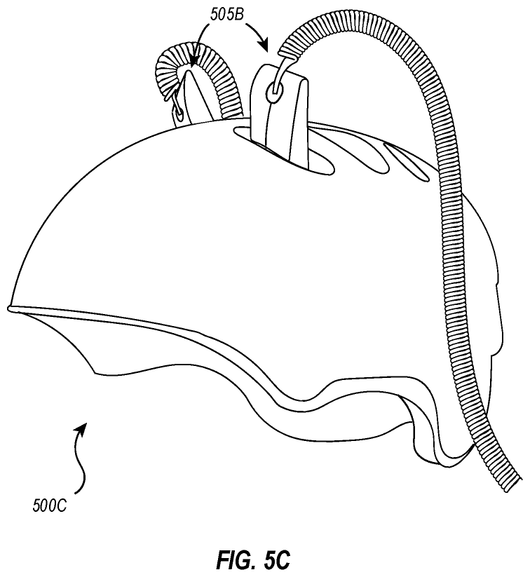

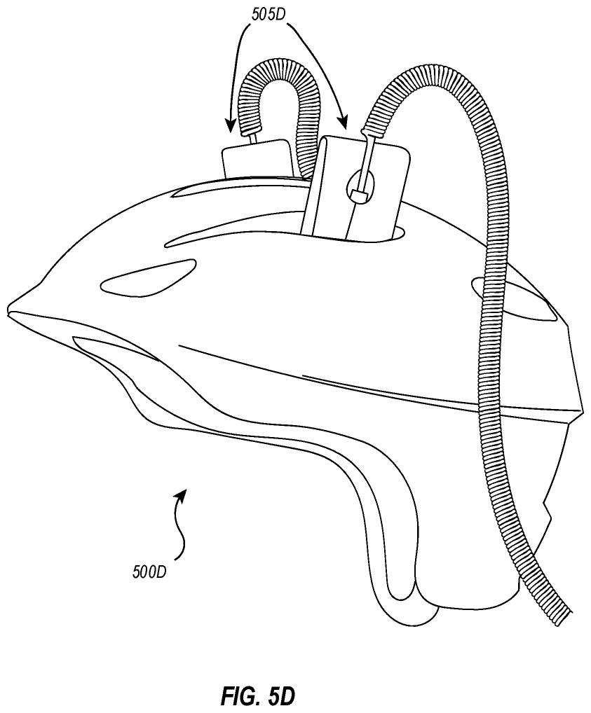

FIGS. 5A-5D illustrate example systems 500A-500D configured for facilitating LLLT on a head of a user. Similar to FIGS. 4A-4D above, FIGS. 5A-5D illustrate various embodiments of head-mounting devices 400 and attachment points 405. FIG. 5A illustrates that the example system 500 includes a head-mounting device 400 as described hereinabove with reference to FIGS. 4A-4D, as well as an LLLT system 100 described hereinabove with reference to FIGS. 1 and 3 (the laser controller 110 of the LLLT system 100 is not shown in FIG. 5). In the example shown in FIG. 5A, the head-mounting device 400 includes at least two attachment points 405, and each attachment point 405 of the head-mounting device 400 is configured to removably secure a laser diode lead 105 of the LLLT system. In some embodiments, each respective attachment point 405 of the head-mounting device 400 is physically separated from one another to allow each respective attachment point 405 to secure a laser diode lead 105 to the head-mounting device 400 and to direct laser light output by the secured laser diode lead 105 toward a respective treatment area on the head of the user (e.g., when the head-mounting device 400 is positioned on the head of the user, as is illustrated in FIG. 5A).

In some implementations, the treatment area(s) that a laser diode lead 105 may be directed toward when secured to an attachment point 405 of a head-mounting device 400 covers only a portion of the scalp of the patient (e.g., rather than covering the entire scalp or most of the scalp of the patient, as is common in devices that emit light not intended to penetrate through the skull of the patient). For instance, in some implementations, a treatment area includes a size that is less than about 50% of the area of the scalp of the user or patient (e.g., or another percentage less than or equal to about 50% of the area of the scalp, such as 45%, 40%, 35%, 30%, 25%, 20%, 15%, 10%, 5%, etc.).

FIGS. 5A-5D shows implementations where the attachment points 405 are configured to facilitate the directing of laser light from laser diodes 105 toward portions of the head or brain along the median plane of the patient's body (e.g., the sagittal plane or left-right plane of the patient's body). For instance, FIGS. 5A-5D show that light from the laser diodes 105 may be directed toward, over and/or around middle portions of the frontal bone of the user, middle portions of the occipital bone of the user, or middle portions between the parietal bones of the user. Such a configuration of the attachment points 405 of the head-mounting device 400 is provided by way of example only and is not limiting of the present disclosure, and a head-mounting device 400 may include any number of attachment points 405 arranged to facilitate the direction of laser light toward any treatment area on a user's head (e.g., to allow LLLT laser light to transmit to different brain tissue areas of users).

Furthermore, FIGS. 5A-5D illustrate an example implementation where the LLLT system 100 includes a plurality of laser diode leads 105 (e.g., two or more laser diode leads), and each separate laser diode lead 105 is secured to a separate attachment point 405 of the head-mounting device 400. In this way, the system 500 of FIGS. 5A-5D may facilitate simultaneous LLLT on separate respective treatment areas on the head of the user wearing the head-mounting device 400 of the system 500.

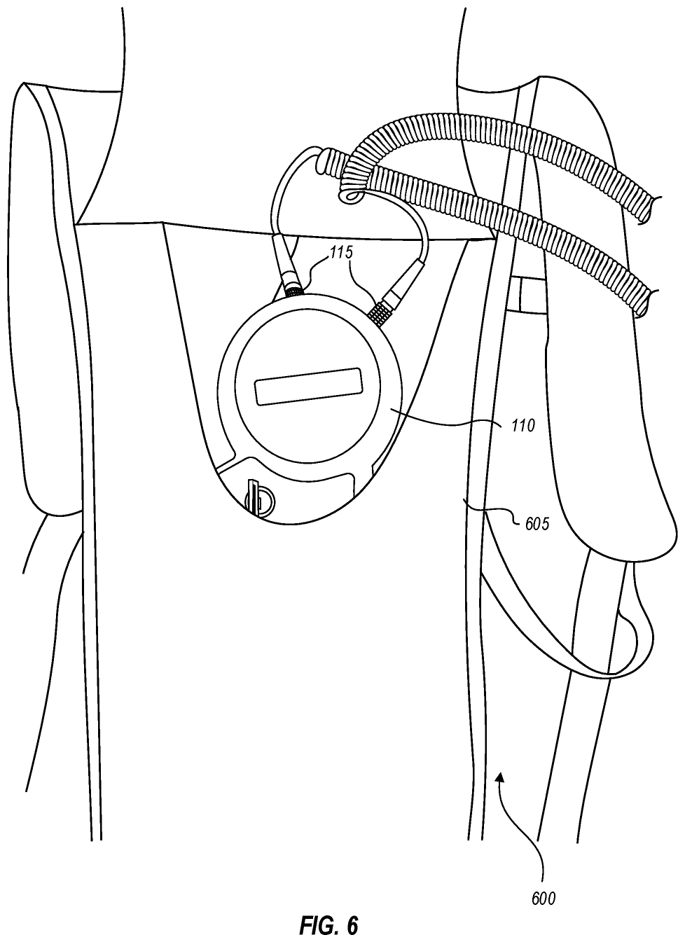

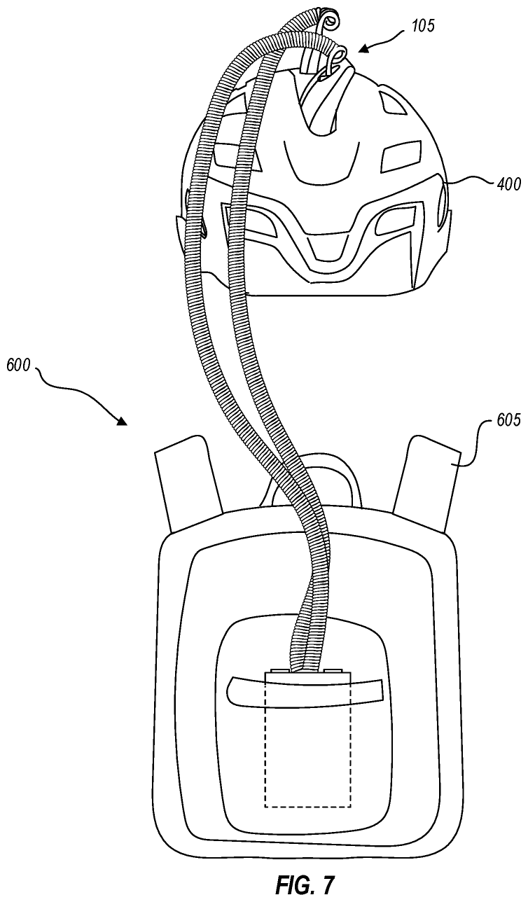

FIGS. 6 and 7 illustrate example implementations of a system 600 configured for facilitating LLLT on a head of a user where the system includes an additional mounting device 605. FIG. 6 shows that an additional mounting device 605 may be configured to receive a laser controller 110 (e.g., for implementations where the laser controller 110 and the laser diode lead(s) 105 is/are not implemented as a single unit). FIGS. 6 and 7 show that the additional mounting device 605 may be configured to be worn by the user wearing the head-mounting device 400 described hereinabove, thereby securing the laser controller 110 to a portion of the body of the user. For example, FIGS. 6 and 7 illustrate an example where the additional mounting device 605 is implemented as a backpack or similar device that is wearable over the shoulders and/or back of the user.

In this regard, when the additional mounting device 605 and the head-mounting device 400 are worn on a user with the laser controller 110 and the laser diode leads 105 disposed therein, respectively, the laser controller 110 and the laser diode leads 105 may be tethered to a single user. Such a configuration may allow the laser controller 110 to remain in proximity to the laser diode leads 105 during use while advantageously allowing the user to move or walk around while receiving LLLT (e.g., without overextending or presenting a safety hazard from any wires extending between the laser controller 110 and the laser diode leads 105).

Although FIGS. 6 and 7 illustrate the additional mounting device 605 implemented for securing the laser controller 110 to the back of the user, it should be noted that an additional mounting device 605 may be configured to secure the laser controller to any portion of the user's body, such as the user's waist, arm(s), chest, thigh, and/or any other suitable body portion(s).

Furthermore, although FIGS. 6 and 7 illustrate implementations that include an additional mounting device 605, one will appreciate, in view of the present disclosure, that a system of the present disclosure for facilitating LLLT on a head of a user may omit an additional mounting device. For instance, a system may be implemented such that the laser controller remains tethered to an environmental object (e.g., a table or cart) such that the user remains substantially tethered to the environmental object when wearing a head-mounting device with laser diode leads extending therefrom toward the laser controller.

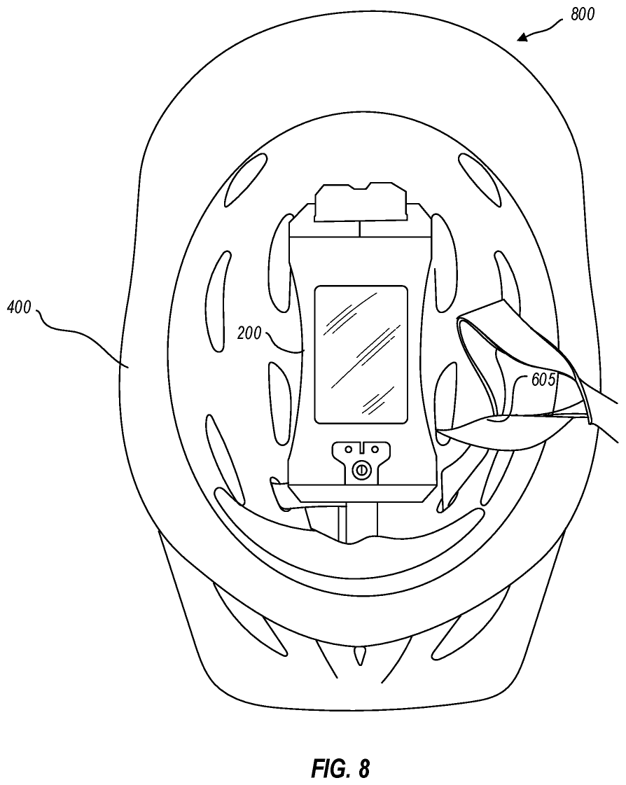

In other instances, the laser controller is also mountable on the head-mounting device (e.g., in addition to the laser diode leads), thereby advantageously providing a system for facilitating LLLT on a head of a user in the form of a single wearable device (e.g., an LLLT system 200 described hereinabove with reference to FIG. 2 may be mountable to a head-mounting device 400). For example, FIG. 8 illustrates a conceptual representation of a system 800 configured for facilitating LLLT on a head of a user where entire the LLLT system 200 (e.g., including the laser diode lead(s) 205 and the laser controller 210, corresponding to the example LLLT system 200 of FIG. 2) is mountable on the head-mounting device 400. For instance, the entire LLLT system 200 may snap or otherwise affix into place on the head-mounting device 400, without any portion of the LLLT system 200 being tethered to a separate device (e.g., an environmental object or additional mounting device). For example, as illustrated in FIG. 8, a mounting device 605 may be in the form of a strap to secure the LLLT system 200 inside the head-mounting device 400. In some instances, as discussed herein, the LLLT system 200 is a conventional LLLT system that can be used independently of the head-mounting device 400. Accordingly, systems of the present disclosure may allow LLLT systems to be used in multiple contexts or ways (e.g., hand-manipulated by a medical practitioner or other user, affixed to a head-mounting device for passive LLLT, etc.).

Furthermore, although the present disclosure focuses, in at least some respects, on implementations where laser diode leads are selectively removable or removably securable to a head-mounting device, systems for facilitating LLLT on a head of a user may be configured in different ways in accordance with the present disclosure. For example, a system for facilitating LLLT on a head of a user may include a head-mounting device with one or more laser diode leads that are fixedly attached thereto, such that the physical arrangement of the laser diode lead(s) on the head-mounting device is not rapidly reconfigurable as described hereinabove.

In some instances, a system includes multiple laser diode leads that are fixedly attached to a head-mounting device, and each separate laser diode lead may be configured to direct light toward a different portion of a user's head when the head-mounting device is worn by the user. The separate laser diode leads may be individually controllable/activatable, such that different LLLT treatment areas, profiles, or configurations may be implemented by the system according to different treatment settings, even though the separate laser diode leads are not rapidly reconfigurable or repositionable on the system. Such treatment settings may be provided by a medical practitioner (e.g., under remote medical care circumstances, such as where in-person meetings with medical practitioners are limited). Such systems may provide a high degree of flexibility and versatility for users desiring to provide or receive LLLT.

It will be appreciated, in view of the present disclosure, embodiments of the present disclosure may implement, comprise, utilize, and/or operate in conjunction or communication with various components to facilitate LLLT on a head of a user (whether such components are explicitly described herein or not).

For example, a system for facilitating any of the disclosed embodiments may be implemented as or include one or more general-purpose or special purpose computing systems, which may take on a variety of forms. For instance, a system may include processor(s), storage, sensor(s), I/O system(s), communication system(s), and/or additional or alternative components.

The processor(s) may comprise one or more sets of electronic circuitries that include any number of logic units, registers, and/or control units to facilitate the execution of computer-readable instructions (e.g., instructions that form a computer program). Such computer-readable instructions may be stored within storage. The storage may comprise physical system memory and may be volatile, non-volatile, or some combination thereof. Furthermore, storage may comprise local storage, remote storage (e.g., accessible via communication system(s) or otherwise), or some combination thereof.

In some implementations, the processor(s) may comprise or be configurable to execute any combination of software and/or hardware components that are operable to facilitate processing using machine learning models or other artificial intelligence-based structures/architectures. For example, processor(s) may comprise and/or utilize hardware components or computer-executable instructions operable to carry out function blocks and/or processing layers configured in the form of, by way of non-limiting example, single-layer neural networks, feed forward neural networks, radial basis function networks, deep feed-forward networks, recurrent neural networks, long-short term memory (LSTM) networks, gated recurrent units, autoencoder neural networks, variational autoencoders, denoising autoencoders, sparse autoencoders, Markov chains, Hopfield neural networks, Boltzmann machine networks, restricted Boltzmann machine networks, deep belief networks, deep convolutional networks (or convolutional neural networks), deconvolutional neural networks, deep convolutional inverse graphics networks, generative adversarial networks, liquid state machines, extreme learning machines, echo state networks, deep residual networks, Kohonen networks, support vector machines, neural Turing machines, and/or others.

In some instances, facilitating the disclosed embodiments may rely at least in part on communication system(s) for receiving data from or facilitating coordinated execution with remote system(s). Remote systems may include, for example, separate systems or computing devices, sensors, and/or others. The communications system(s) may comprise any combination of software or hardware components that are operable to facilitate communication between on-system components/devices and/or with off-system components/devices. For example, the communications system(s) may comprise ports, buses, or other physical connection apparatuses for communicating with other devices/components. Additionally, or alternatively, the communications system(s) may comprise systems/components operable to communicate wirelessly with external systems and/or devices through any suitable communication channel(s), such as, by way of non-limiting example, Bluetooth, ultra-wideband, WLAN, infrared communication, and/or others.

In some instances, facilitating the disclosed embodiments may rely at least in part on data obtained via sensor(s). Such sensor(s) may comprise any system or device for capturing or measuring data representative of perceivable phenomena. By way of non-limiting example, the sensor(s) may comprise one or more image sensors, microphones, thermometers, barometers, magnetometers, accelerometers, gyroscopes, and/or others.

Furthermore, in some instances, facilitating the disclosed embodiments may rely at least in part on I/O system(s). I/O system(s) may include any type of input or output device such as, by way of non-limiting example, a touch screen, a mouse, a keyboard, a controller, and/or others, without limitation.

Disclosed embodiments may comprise or utilize a special purpose or general-purpose computer including computer hardware, as discussed in greater detail below. Disclosed embodiments also include physical and other computer-readable media for carrying or storing computer-executable instructions and/or data structures. Such computer-readable media can be any available media that can be accessed by a general-purpose or special-purpose computer system. Computer-readable media that store computer-executable instructions in the form of data are “physical computer storage media” or a “hardware storage device.” Computer-readable media that merely carry computer-executable instructions without storing the computer-executable instructions are “transmission media.” Thus, by way of example and not limitation, the current embodiments can comprise at least two distinctly different kinds of computer-readable media: computer storage media and transmission media.

Computer storage media (aka “hardware storage device”) are computer-readable hardware storage devices, such as RANI, ROM, EEPROM, CD-ROM, solid state drives (“SSD”) that are based on RANI, Flash memory, phase-change memory (“PCM”), or other types of memory, or other optical disk storage, magnetic disk storage or other magnetic storage devices, or any other medium that can be used to store desired program code means in the form of computer-executable instructions, data, or data structures and that can be accessed by a general-purpose or special-purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links which can be used to carry program code in the form of computer-executable instructions or data structures, and which can be accessed by a general purpose or special purpose computer. Combinations of the above are also included within the scope of computer-readable media.

Further, upon reaching various computer system components, program code means in the form of computer-executable instructions or data structures can be transferred automatically from transmission computer-readable media to physical computer-readable storage media (or vice versa). For example, computer-executable instructions or data structures received over a network or data link can be buffered in RANI within a network interface module (e.g., a “NIC”), and then eventually transferred to computer system RANI and/or to less volatile computer-readable physical storage media at a computer system. Thus, computer-readable physical storage media can be included in computer system components that also (or even primarily) utilize transmission media.

Computer-executable instructions comprise, for example, instructions and data which cause a general-purpose computer, special purpose computer, or special purpose processing device to perform a certain function or group of functions. The computer-executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Disclosed embodiments may comprise or utilize cloud computing. A cloud model can be composed of various characteristics (e.g., on-demand self-service, broad network access, resource pooling, rapid elasticity, measured service, etc.), service models (e.g., Software as a Service (“SaaS”), Platform as a Service (“PaaS”), Infrastructure as a Service (“IaaS”), and deployment models (e.g., private cloud, community cloud, public cloud, hybrid cloud, etc.).

Those skilled in the art will appreciate that the invention may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, pagers, routers, switches, wearable devices, and the like. The invention may also be practiced in distributed system environments where multiple computer systems (e.g., local and remote systems), which are linked through a network (either by hardwired data links, wireless data links, or by a combination of hardwired and wireless data links), perform tasks. In a distributed system environment, program modules may be located in local and/or remote memory storage devices.

Alternatively, or in addition, the functionality described herein can be performed, at least in part, by one or more hardware logic components. For example, and without limitation, illustrative types of hardware logic components that can be used include Field-programmable Gate Arrays (FPGAs), Program-specific Integrated Circuits (ASICs), Application-specific Standard Products (ASSPs), System-on-a-chip systems (SOCs), Complex Programmable Logic Devices (CPLDs), central processing units (CPUs), graphics processing units (GPUs), and/or others.

As used herein, the terms “executable module,” “executable component,” “component,” “module,” or “engine” can refer to hardware processing units or to software objects, routines, or methods that may be executed on one or more computer systems. The different components, modules, engines, and services described herein may be implemented as objects or processors that execute on one or more computer systems (e.g., as separate threads).

One will also appreciate how any feature or operation disclosed herein may be combined with any one or combination of the other features and operations disclosed herein. Additionally, the content or feature in any one of the Figures may be combined or used in connection with any content or feature used in any of the other figures. In this regard, the content disclosed in any one Figure is not mutually exclusive and instead may be combinable with the content from any of the other figures.

The present invention may be embodied in other specific forms without departing from its spirit or characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A system configured for facilitating low-level laser therapy (LLLT) on a head of a user, comprising:
an LLLT system, comprising:
a laser diode lead configured to output laser light suitable for LLLT; and
a laser controller configured to provide power to and control operation of the laser diode lead; and
a head-mounting device wearable on a head of a user, the head-mounting device comprising an attachment point, the attachment point being configured to secure the laser diode lead and to direct laser light output by the laser diode lead toward a treatment area on a head of a user.

2. The system of claim 1, wherein the LLLT system is configured to output laser light for penetrating through a skull of the user to reach brain tissue of the user.

3. The system of claim 1, wherein the LLLT system is configured output laser light comprising one or more wavelengths within the near-infrared region.

4. The system of claim 1, wherein the LLLT system is configured to output laser light comprising one or more wavelengths within a range of about 780 nm to about 840 nm.

5. The system of claim 1, wherein the LLLT system omits light-emitting diodes for administering LLLT to the treatment area on the head of the user.

6. The system of claim 1, wherein the treatment area on the head of the user comprises a size that is less than 50% of an area of a scalp of the user.

7. The system of claim 1, wherein the attachment point is positioned on the head-mounting device to orient the laser diode lead to direct laser light toward a middle portion of a frontal bone, occipital bone, or between parietal bones of the user.

8. The system of claim 1, wherein the attachment point is configured to selectively and removably secure the laser diode lead to the head-mounting device.

9. The system of claim 1, wherein the attachment point comprises a slot sized to receive the laser diode lead.

10. The system of claim 1, wherein the head-mounting device comprises a plurality of attachment points, each attachment point of the plurality of attachment points being configured to removably secure the laser diode lead to direct laser light output by the laser diode lead toward a respective treatment area on the head of the user.

11. The system of claim 10, wherein the LLLT system comprises a plurality of laser diode leads configured to simultaneously secure to separate attachment points of the plurality of attachment points of the head-mounting device to facilitate simultaneous LLLT on separate respective treatment areas on the head of the user.

12. The system of claim 11, wherein the LLLT system comprises a conventional LLLT system not specially manufactured for mounting to a head-mounting device.

13. The system of claim 1, wherein the laser controller is mountable on the head-mounting device.

14. The system of claim 1, further comprising an additional mounting device configured for receiving the laser controller to secure the laser controller to a portion of a body of the user.

15. A system configured for facilitating low-level laser therapy (LLLT) on a head of a user, comprising:
an LLLT system, comprising:
a laser diode lead configured to output laser light comprising one or more wavelengths within a range of about 780 nm to about 840 nm; and
a laser controller configured to provide power to and control operation of the laser diode lead; and
a head-mounting device wearable on a head of a user, the head-mounting device comprising an attachment point, the attachment point being configured to secure the laser diode lead and to direct laser light output by the laser diode lead toward a treatment area on a head of a user,
wherein the attachment point is positioned on the head-mounting device to orient the laser diode lead to direct laser light toward a middle portion of a frontal bone, occipital bone, or between parietal bones of the user, and
wherein the attachment point is configured to selectively and removably secure the laser diode lead to the head-mounting device.

16. The system of claim 15, wherein the head-mounting device comprises a plurality of attachment points, each attachment point of the plurality of attachment points being configured to removably secure the laser diode lead to direct laser light output by the laser diode lead toward a respective treatment area on the head of the user.

17. The system of claim 16, wherein the LLLT system comprises a plurality of laser diode leads configured to simultaneously secure to separate attachment points of the plurality of attachment points of the head-mounting device to facilitate simultaneous LLLT on separate respective treatment areas on the head of the user.

18. The system of claim 15, wherein the laser controller is mountable on the head-mounting device.

19. The system of claim 15, further comprising an additional mounting device configured for receiving the laser controller to secure the laser controller to a portion of a body of the user.

20. A method of providing low-level laser therapy (LLLT) on a head of a user, the method comprising:
providing an LLLT system that includes a laser diode lead configured to output laser light suitable for LLLT and a laser controller configured to provide power to and control operation of the laser diode lead;
providing a head-mounting device wearable on a head of a user, the head-mounting device comprising an attachment point, the attachment point being configured to secure the laser diode lead and to direct laser light output by the laser diode lead toward a treatment area on a head of a user;
positioning the LLLT system within the head-mounting device; and
operating the LLLT system to output laser light directed to the head of the user.
